(19) United States
(12) Patent Application Publication
(10) Pub. No.: US 2017/0176437 A1
(43) Pub. Date: Jun. 22, 2017

(54) Method of diagnosing and monitoring bladder cancer and melanoma

(71) Applicant: Francis Eugene Wilkinson, Frankfort, IN (US)

(72) Inventor: Francis Eugene Wilkinson, Frankfort, IN (US)

(21) Appl. No.: 14/970,972

(22) Filed: Dec. 16, 2015

(51) Int. Cl. G01N 33/574

(52) U.S. Cl. CPC G01N 33/57407; G01N 33/5743; G01N 2333/90245; G01N 2333/705; G01N 2800/56

(57) ABSTRACT

The present invention provides a method for the diagnosing and monitoring of melanoma

### FIELD OF THE INVENTION

This invention relates, in part, to newly developed assays for diagnosing and monitoring melanoma.

### BACKGROUND OF THE INVENTION

It has been estimated that in the United States in 2015 that over 1,650,000 new cases of cancer will be diagnosed and that there will be over 589,000 deaths due to cancer. It is estimated that melanoma will account for 9,940 of the cancer deaths in the United States. See “Cancer Facts & Figures 2015.” p. 4 American Cancer Society (2015).

There are at least three medical conditions that present a pressing need for sensitive diagnostic tests for melanoma. These include:
1. As an early detection marker. The purpose of early detection markers is to detect the cancer while it is localized. Localized tumors are generally small and usually can be surgically removed before the cancer metastasizes. Cancer that has metastasized, even to nearby organs, is considered to be incurable.
   The PSA test for prostate cancer is the only FDA-approved early detection marker for any cancer. The PSA test has revolutionized the diagnosis of prostate cancer and has been instrumental in lowering the age-adjusted death rate by fifty percent since 1992. See Howlader et al., “Cancer of the Prostate (Invasive).” Section 23 SEER Cancer Statistics Review, 1975-2012, p. 6 (April 2015). The PSA test has an actual clinical sensitivity of 53.2% and 69.9% for stages A and B, respectively, indicating that the PSA test cannot identify all cases of early stage prostate cancer. See “Architect System Total PSA.” Abbott Laboratories p. 6 (June, 2007).
   The introduction of an early detection marker for melanoma could reduce the deaths due to melanoma by over 4,000. Early detection tests for the top twenty cancers could reduce the annual number of deaths due to cancer by nearly 300,000.
2. As a staging marker. Staging markers assist clinicians in determining whether the cancer is localized or has metastasized. The five year survival data of bladder cancer patients diagnosed with localized disease illustrates the problem as 30.1% of these patients die within five years of diagnosis. See Howlader et al., “Cancer of the Urinary Bladder (Invasive and In Situ).” Section 23 SEER Cancer Statistics Review, 1975-2012, p. 8 (April 2015). This data suggests that many of these bladder cancer patients actually had metastatic disease when the initial diagnosis was that they had localized disease. The treatments for localized and metastatic disease are quite different, and proper staging is critical to a successful treatment.
3. As a monitoring marker. Monitoring markers are a real time analytic of the growth status of previously diagnosed cancer. Monitoring markers are used to determine if the cancer shows no evidence of disease, is stable, is responding to treatment, or is progressing. The test result can be critical in determining the proper treatment.

### Melanoma

The American Cancer Society estimates that in the United States in 2015 that 73,870 people will be diagnosed with melanoma and that 9,940 people will die of melanoma. See “Melanoma Skin Cancer.” p. 6 American Cancer Society (Nov. 10, 2015).

There are more than 996,000 people in the United States who have been diagnosed with melanoma and are living with the disease. See Howlader et al., “Melanoma of the Skin.” SEER Cancer Statistics Review, 1975-2012 Section 16 p. 21 (April 2015). Melanoma affects both sexes; twice as many men die of melanoma than women. Melanoma is usually diagnosed while the tumor is localized, 84% of all cases. Nine percent of the cases are diagnosed as regional, four percent as distant, and three percent are diagnosed as unstaged. Melanoma patients whose tumor was diagnosed as localized have a five year survival rate of 98.3%. Those with regional and distant disease at the time of diagnosis have five-year survival rates of 63.0% and 16.6%, respectively. See Howlader et al., “Melanoma of the Skin.” SEER Cancer Statistics Review, 1975-2012 Section 16 p. 8 (April 2015).

Melanoma is an increasingly prevalent health problem in the United States due to the intentional exposure to ultraviolet radiation via the sun and tanning salons. Melanoma originates in melanocytes. Melanocytes exist as individual cells at the basal level of the epidermis over virtually all exposed portions of the body. Melanocytes produce melanosomes which synthesize the dark brown to black skin pigment eumelanin. The function of eumelanin is to protect the skin from the deleterious effects of ultraviolet radiation. Melanosomes in tow are transported through the dendrites of melanosomes and are taken up by the microvilli of the keratinocytes in the epidermis. The melanosomes are transported to a position between the nucleus and the outer layer of the epidermis of the recipient cell so that eumelanin is in a position to protect the recipient cell's nucleus and DNA from ultraviolet light. See Ando et al., J. Invest. Dermatol. 132.4 (2012): 1222-1229.

Tyrosinase related protein 1, TYRP1, is involved in the synthesis of eumelanin from the amino acid L-tyrosine, but its function in this process is unclear. TYRP1 may catalyze one or two of the enzymatic reactions in the pathway, or it may be involved in the transport of eumelanin. See Hearing et al., FASEB J. 5.14 (1991):2902-2909.

Expression profiling of melanoma biopsy tissue found that TYRP1 is the gene that correlates most strongly with poor survival of melanoma with a p of 0.00004. Four of the next fourteen genes most highly associated with poor melanoma survival, SILV, DCT, OCA2, and TYR are also melanogenesis-related genes. See Journe et al., Br. J. Cancer. 105.11 (2011):1726-1732.

To date, no increase of TYRP1 protein or its peptides in melanoma relative to normal skin tissue has been found. Nor has there been any report of increased TYRP1 protein or peptide in the blood or any other bodily fluid of melanoma patients relative to healthy individuals.

According to the American Cancer Society there are no blood tests for diagnosing, staging, or monitoring melanoma. See “Melanoma Skin Cancer.” American Cancer Society p. 21 (Nov. 10, 2015).

### BRIEF DESCRIPTION OF THE SEVERAL VIEWS OF THE DRAWING

FIG. 1, shows the results of a dot blot showing the plasma level of TYRP 1 in two newly-diagnosed patients with melanoma and five Black and five White healthy blood donors.

### DETAILED DESCRIPTION OF CERTAIN EMBODIMENTS OF THE INVENTION

The present invention relates to diagnostic assays, both quantitative and qualitative for detecting levels of TYRP1 or its polypeptides in bodily fluids including empirical determination of normal and abnormal levels. Thus, for instance, a diagnostic assay in accordance with the invention of detecting over-expression of TYRP1 in bodily fluids of individuals who may have melanoma compared to levels in healthy individuals. Further, the present method of quantifying the TYRP1 levels is particularly useful for discriminating between melanoma and other diseases of the skin since some of the other methods for discriminating melanoma and other ailments of the skin require a biopsy and examination by a pathologist. The current invention could be performed in a physician's office and have a reliable result within three hours. Assay techniques that can be used to determine levels of gene expression of antigens like TYRP1, of the present invention, in a sample derived from a host are well-known to those of skill in the art. Such assay methods include, but are not limited to, radioimmunoassay, competitive binding assays, Western Blot analyses, dot blots, and ELISAs.

Without limiting the type of assay in the instant invention, ideally for a quantitative diagnostic assay a positive result indicating the disease is one in which blood levels are higher than three standard deviations above the mean level for a normal healthy population of individuals (99.865% of the population). A best case result would yield 100% clinical sensitivity and 100% clinical specificity, which means that the test correctly identifies every individual with the disease as having the disease and also identifying 100% of those who do not as not having the disease, respectively.

The present invention is further described by the following examples. These examples are provided solely to illustrate the invention by reference to specific embodiments. These examples, while illustrating certain specific aspects of the invention, do not portray the limitations or circumscribe the scope of the disclosed invention.

### Example 1

Demonstrating that the blood level of TYRP1 is elevated in melanoma patients relative to healthy individuals.

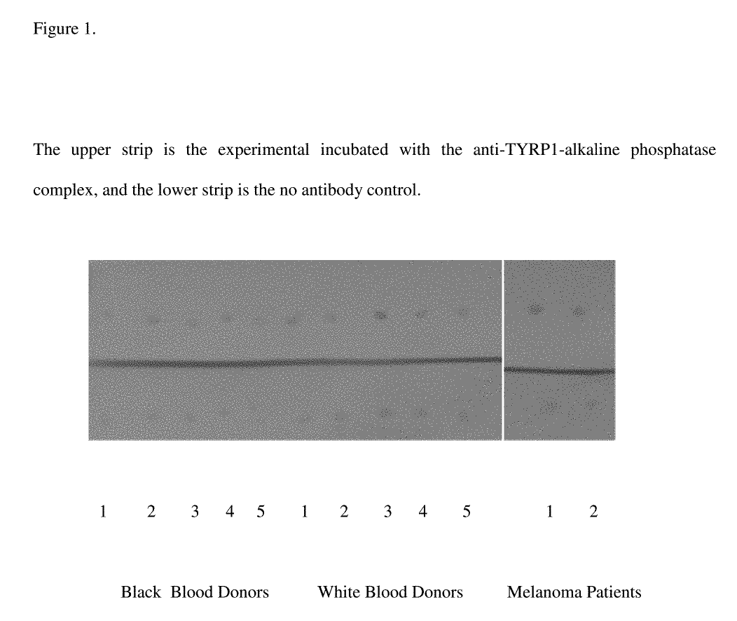

FIG. 1, shows the results of a dot blot showing the plasma level of TYRP 1 in two patients with melanoma and in ten healthy blood donors.

The two melanoma patients were newly diagnosed White males with melanoma, and the blood was drawn prior to any treatment for melanoma. The thickness of the tumors were 10 mm and 4 mm for patients #1 and #2, respectively. Ten healthy male blood donors were used as the control population. Five were Black, and five were White. Black males were included to determine if the blood level of TYRP1 is strongly associated with race.

A small volume of patient and control plasma was diluted, and identical volumes of each were spotted onto two strips of nitrocellulose. The other binding sites on the nitrocellulose were covered by incubating the nitrocellulose in a solution of bovine serum albumin.

A mouse monoclonal antibody to TYRP1 was purchased from GeneTex. This antibody had been shown to react with melanocytes, melanoma and nevi and that it does not react with carcinomas or sarcomas. See “TRP1 antibody [Ta99] GTX11780 Datasheet.” p. 1 GeneTex (Accessed January, 2012).

The antibody was biotinylated, then reacted with streptavidin-Alkaline-Phosphatase to produce an antibody to TYRP1 with the Alkaline-Phosphatase reporter attached. The experimental and control strips were put into incubation media with or without the anti-TYRP1-Alkaline Phosphatase complex, respectively. Both strips of nitrocellulose were incubated identically. Both were washed, then put into buffer containing the substrate for alkaline phosphatase for visualizing TYRP1.

The data in FIG. 1 show that the blood level of TYRP1 in both newly diagnosed melanoma patients is higher than the blood level of nine of the ten plasma samples from the healthy blood donors, whether Black or White.

These results indicate that the level of TYRP1 in human blood, or other human bodily fluids, could be used in diagnostic assays for distinguishing individuals who have melanoma from those who do not.

Thus, TYRP1 could be useful in diagnosing and monitoring of melanoma.

### REFERENCES

Ando et al., “Melanosomes Are Transferred from Melanocytes to Keratinocytes through the Processes of Packaging, Release, Uptake, and Dispersion.” J. Invest. Dermatol. 132.4 (2012): 1222-1229.
“Architect System Total PSA.” pp. 1-8 Abbott Laboratories (June, 2007).
“Cancer Facts and Figures 2015” pp. 1-56 American Cancer Society (Nov. 10, 2015).
Hearing et al., “Enzymatic Control of Pigmentation in Mammals.” FASEB J. 5.14 (1991):2902-2909.
Howlader et al., “Melanoma of the Skin.” SEER Cancer Statistics Review, 1975-2012, Section 16 pp. 1-27 National Cancer Institute. Bethesda, Md., http://seer.cancer.gov/csr/19752012/, based on November 2014 SEER data submission, posted to the SEER web site, April 2015.
Howlader et al., “Cancer of the Prostate (Invasive).” SEER Cancer Statistics Review, 1975-2012, Section 23 pp. 1-22 National Cancer Institute. Bethesda, Md., http://seer.cancer.gov/csr/19752012/, based on November 2014 SEER data submission, posted to the SEER web site, April 2015.
Howlader et al., “Cancer of the Urinary Bladder Section (Invasive and In Situ).” SEER Cancer Statistics Review, 1975-2012, Section 27 pp. 1-26 National Cancer Institute. Bethesda, Md., http://seer.cancer.gov/csr/19752012/, based on November 2014 SEER data submission, posted to the SEER web site, April 2015.
Journe et al., “TYRP1 mRNA Expression in Malignant Melanoma Metastases Correlates with Clinical Outcome.” Br. J. Cancer. 105.11 (2011):1726-1732.
“Melanoma Skin Cancer.” p. 62 American Cancer Society (Nov. 10, 2015).
“TRP1 antibody [Ta99] GTX11780 Datasheet.” p. 1 GeneTex (Accessed January, 2012).

What is claimed is:

1. A method for diagnosing, monitoring, and staging of melanoma comprising: the use of an analytical method such as, but not limited to, ELISA, radioimmunoassay, competitive binding assay, Western blot, or dot blot, to measure the quantity of TYRP1 in any human bodily fluid wherein TYRP1 polypeptide level higher than three standard deviations above normal control is associated with the presence of melanoma
